(19) United States
(12) Patent Application Publication
(10) Pub. No.: US 2012/0237581 A1
(43) Pub. Date: Sep. 20, 2012

(54) Method Of Producing A Silicone Powder-Containing Oil Composition

(76) Inventors: Tadashi Takimoto, Ichihara-shi (JP); Yoshitsugu Morita, Ichihara-shi (JP)

(21) Appl. No.: 13/509,023

(22) PCT Filed: Nov. 10, 2010
(86) PCT No.: PCT/JP2010/070423

§ 371 Date: May 10, 2012

(30) Foreign Application Priority Data
Nov. 13, 2009 (JP) JP 2009-259741

(51) Int. Cl. C08L 83/07; C10M 155/02; A61K 8/02; A61K 9/00; A61K 8/92; A61K 47/30

(52) U.S. Cl. 424/401; 514/772.3; 424/400; 508/577; 524/588

(57) ABSTRACT

The present invention relates to a method of producing a silicone powder-containing oil composition, characterized by removing the water from a water-based emulsion of a silicone powder-containing oil composition while stirring this emulsion under reduced pressure with a stirring apparatus that has at least a stirring means that rotates at low speed along the inner wall of a container and a stirring means that rotates at high speed in the interior of the container.

### TECHNICAL FIELD

The present invention relates to a method of producing a silicone powder-containing oil composition.

### BACKGROUND ART

Patent Documents 1 to 5 teach that a silicone powder-containing oil composition can be produced by removing the water from a water-based emulsion that contains a silicone powder in oil droplets that are dispersed in the water. These emulsions contain a surfactant. The surfactant is altered by the application of heat and thereby produces odor. For this, it is taught that these emulsions cannot be dried by heating and the use of air drying is applicable.

However, it is difficult with these methods to efficiently produce silicone powder-containing oil compositions. As a consequence, methods of water removal while stirring under reduced pressure have been examined for removal of the water from the aforementioned emulsions, but it has been found in these methods that the silicone powder aggregates and sticks to the stirring blades, and/or the oil composition sticks to the stirring blades, which impairs the efficient removal of the water and impairs the efficient production of an oil composition in which the silicone powder is uniformly dispersed in the oil.

| | |
|---|---|
| [Patent Document 1] | JP 2000-281523 A |
| [Patent Document 2] | JP 2000-281903 A |
| [Patent Document 3] | JP 2001-139416 A |
| [Patent Document 4] | JP 2001-139819 A |
| [Patent Document 5] | JP 2002-249588 A |

### SUMMARY OF INVENTION

### Technical Problems to be Solved

An object of the present invention is to provide a method that efficiently produces, by the removal of water from the water-based emulsion of a silicone powder-containing oil composition, an oil composition in which the silicone powder is uniformly dispersed.

### Solution to Problems

The method of the present invention for producing a silicone powder-containing oil composition is characterized by removing the water from a water-based emulsion of a silicone powder-containing oil composition while stirring this emulsion under reduced pressure with a stirring apparatus that has at least a stirring means that rotates at low speed along the inner wall of a container and a stirring means that rotates at high speed in the interior of the container.

### Advantageous Effects of Invention

The production method of the present invention is characteristically able to efficiently produce, by the removal of water from the water-based emulsion of a silicone powder-containing oil composition, an oil composition in which the silicone powder is uniformly dispersed.

### BRIEF DESCRIPTION OF THE DRAWINGS

FIG. 1 is a cross-sectional drawing that shows an example of a stirring apparatus used by the production method of the present invention.

### BEST MODE FOR CARRYING OUT THE INVENTION

The water-based emulsion of a silicone powder-containing oil composition that is used by the present invention is a water-based emulsion in which a silicone powder and an oil are dispersed in water, and preferably is a water-based emulsion that contains a silicone powder in oil droplets that are themselves emulsified in the water. The form of this silicone powder is preferably an elastomeric form such as a gel, rubber, and so forth. The average particle size of this silicone powder is preferably in the range from 0.05 to 100 μm, more preferably in the range from 0.1 to 100 μm, and particularly preferably in the range from 0.1 to 50 μm. Such a silicone powder is preferably provided by the crosslinking reaction of a crosslinkable silicone composition, for example, based on the hydrosilylation reaction, a condensation reaction, an organoperoxide-mediated radical reaction, or a high energy radiation-mediated radical reaction, and is particularly preferably provided by crosslinking based on the hydrosilylation reaction or a condensation reaction.

Hydrosilylation reaction-crosslinkable silicone compositions can be exemplified by a composition comprising at least (A) an organopolysiloxane that has at least two alkenyl groups in each molecule, (B) an organopolysiloxane that has at least two silicon-bonded hydrogen atoms in each molecule, and (C) a hydrosilylation reaction catalyst.

The alkenyl group in component (A) can be exemplified by vinyl, allyl, butenyl, pentenyl, and hexenyl with vinyl being particularly preferred. The non-alkenyl silicon-bonded organic groups in component (A) can be exemplified by monovalent hydrocarbyl groups, e.g., alkyl groups such as methyl, ethyl, propyl, butyl, and so forth; cycloalkyl groups such as cyclopentyl, cyclohexyl, and so forth; aryl groups such as phenyl, tolyl, xylyl, and so forth; aralkyl groups such as benzyl, phenethyl, 3-phenylpropyl, and so forth; and halogenated hydrocarbyl groups such as 3-chloropropyl, 3,3,3-trifluoropropyl, and so forth. The molecular structure of component (A) can be exemplified by straight chain, cyclic, network, and partially branched straight chain, and straight chain and partially branched straight chain are preferred when the formation of an elastomeric silicone powder is sought. Component (A) has a viscosity at 25° C. preferably in the range from 20 to 100,000 mPa·s and particularly preferably in the range from 20 to 10,000 mPa·s.

The non-hydrogen silicon-bonded organic groups in component (B) can also be exemplified by monovalent hydrocarbyl groups excluding the same alkenyl group as cited above. The molecular structure of component (B) can be exemplified by straight chain, cyclic, network, and partially branched straight chain. Component (B) has a viscosity at 25° C. preferably in the range from 1 to 10,000 mPa·s. Component (B) is incorporated in an amount sufficient to bring about the crosslinking of the composition under consideration, but its amount of incorporation is not otherwise particularly limited. In specific terms, component (B) is preferably incorporated in the range from 0.3 to 200 weight parts per 100 weight parts component (A).

Component (C) is a hydrosilylation reaction catalyst and is used to promote the crosslinking reaction in the composition under consideration. Component (C) is particularly preferably a platinum catalyst. This platinum catalyst can be exemplified by chloroplatinic acid, alcohol solutions of chloroplatinic acid, olefin complexes of platinum, alkenylsiloxane complexes of platinum, platinum black, and platinum supported on silica. Component (C) is incorporated in a quantity sufficient to promote the crosslinking reaction in the composition under consideration, but its quantity of incorporation is not otherwise particularly limited. In those instances in which a platinum catalyst is used for component (C), component (C) is preferably incorporated in an amount that provides from 1×10⁻⁷ to 1×10⁻³ weight part platinum metal in component (C) for each 100 weight parts of the total of components (A) and (B).

When crosslinking of this composition is to be undertaken with the composition dispersed in water, component (C) may be incorporated in the composition in advance and the composition may then be crosslinked, or the crosslinkable silicone composition, excluding component (C), may be dispersed in water and component (C) may thereafter be added to the water and the composition may then be crosslinked. In the latter case, the use is preferred of a water-based dispersion in which component (C) has been dispersed to an average particle size of not more than 1 μm.

The condensation reaction-crosslinkable silicone compositions can be exemplified by a composition comprising at least (D) an organopolysiloxane that has at least two silicon-bonded hydroxyl groups or hydrolyzable groups in each molecule wherein the hydrolyzable group can be exemplified by the alkoxy group, oxime group, acetoxy group, aminoxy group, and so forth, (E) a silane-type crosslinking agent that has at least three silicon-bonded hydrolyzable groups, e.g., the alkoxy group, oxime group, acetoxy group, aminoxy group, and so forth, in each molecule, and (F) a condensation reaction catalyst such as an organotin compound, an organotitanium compound, and so forth.

The alkoxy group for component (D) can be exemplified by methoxy, ethoxy, and methoxyethoxy. The oxime group for component (D) can be exemplified by the dimethyl ketoxime group and the methyl ethyl ketoxime group. The other silicon-bonded organic groups in component (D) can be exemplified by monovalent hydrocarbyl groups, e.g., alkyl groups such as methyl, ethyl, propyl, butyl, and so forth; cycloalkyl groups such as cyclopentyl, cyclohexyl, and so forth; alkenyl groups such as vinyl, allyl, butenyl, pentenyl, and hexenyl; aryl groups such as phenyl, tolyl, xylyl, and so forth; aralkyl groups such as benzyl, phenethyl, 3-phenylpropyl, and so forth; and halogenated hydrocarbyl groups such as 3-chloropropyl, 3,3,3-trifluoropropyl, and so forth. The molecular structure of component (D) can be exemplified by straight chain, cyclic, network, and partially branched straight chain, and straight chain and partially branched straight chain are preferred when the formation of an elastomeric silicone powder is sought. Component (D) has a viscosity at 25° C. preferably in the range from 20 to 100,000 mPa·s and particularly preferably in the range from 20 to 10,000 mPa·s.

The alkoxy group and oxime group for component (E) can be exemplified by the same groups as already provided above. Component (E) can be exemplified by methyltrimethoxysilane, vinyltrimethoxysilane, methyltrioximesilane, and vinyltrioximesilane. Component (E) is incorporated in an amount sufficient to bring about the crosslinking of the composition under consideration, but its amount of incorporation is not otherwise particularly limited. In specific terms, component (E) is preferably incorporated in the range from 0.3 to 200 weight parts per 100 weight parts component (D).

Component (F) is a condensation reaction catalyst and is used to promote the crosslinking reaction in the composition under consideration. It can be specifically exemplified by dibutyltin dilaurate, dibutyltin diacetate, tin octenoate, dibutyltin dioctate, tin laurate, tetrabutyl titanate, tetrapropyl titanate, and dibutoxybis(ethyl acetoacetato)titanium. Component (F) is incorporated in an amount sufficient to promote the crosslinking reaction of the composition under consideration, but its amount of incorporation is not otherwise particularly limited. In specific terms, component (F) is preferably incorporated in the range from 0.01 to 5 weight parts per 100 weight parts component (D) and particularly preferably in the range from 0.05 to 2 weight parts per 100 weight parts component (D).

There are no particular limitations on the oil, but a silicone oil or a silicon-free organic oil is preferred. The viscosity of this oil at 25° C. is preferably in the range from 1 to 100,000,000 mPa·s and particularly preferably is in the range from 2 to 10,000,000 mPa·s.

The silicone oil can be exemplified by a dimethylpolysiloxane that is endblocked at both molecular chain terminals by the trimethylsiloxy group, a methylphenylpolysiloxane that is endblocked at both molecular chain terminals by the trimethylsiloxy group, a dimethylsiloxane-methylphenylsiloxane copolymer that is endblocked at both molecular chain terminals by the trimethylsiloxy group, a dimethylsiloxane-methyl(3,3,3-trifluoropropyl)siloxane copolymer that is endblocked at both molecular chain terminals by the trimethylsiloxy group, cyclic dimethylsiloxanes, cyclic methylphenylsiloxanes, a dimethylpolysiloxane that is endblocked at both molecular chain terminals by the dimethylvinylsiloxy group, a dimethylsiloxane-methylvinylsiloxane copolymer that is endblocked at both molecular chain terminals by the dimethylvinylsiloxy group, a methylvinylpolysiloxane that is endblocked at both molecular chain terminals by the trimethylsiloxy group, cyclic methylvinylsiloxanes, a dimethylpolysiloxane that is endblocked at both molecular chain terminals by the silanol group, a methylphenylpolysiloxane that is endblocked at both molecular chain terminals by the silanol group, a dimethylsiloxane-methylphenylsiloxane copolymer that is endblocked at both molecular chain terminals by the silanol group, and a dimethylsiloxane-methyl(3,3,3-trifluoropropyl)siloxane copolymer that is endblocked at both molecular chain terminals by the silanol group, and can also be exemplified by a dimethylpolysiloxane that is endblocked at both molecular chain terminals by the trimethoxysiloxy group, a methylphenylpolysiloxane that is endblocked at both molecular chain terminals by the trimethoxysiloxy group, a dimethylsiloxane-methylphenylsiloxane copolymer that is endblocked at both molecular chain terminals by the trimethoxysiloxy group, and a dimethylsiloxane-methyl(3,3,3-trifluoropropyl)siloxane copolymer that is endblocked at both molecular terminals by the trimethoxysiloxy group.

In those instances in which the water-based emulsion of a silicone powder-containing silicone oil composition is produced by the preliminary incorporation of the silicone oil in the crosslinkable silicone composition, its is preferred to use a silicone oil that does not participate in the composition's crosslinking reaction. In specific terms, when the crosslinking reaction is the hydrosilylation reaction, the silicone oil molecule is to be free of the alkenyl group or silicon-bonded hydrogen; when the crosslinking reaction is a condensation reaction, the silicone oil molecule is to be free of the silanol group, silicon-bonded hydrogen, and silicon-bonded hydrolyzable groups.

The silicon-free organic oil can be exemplified by fats and oils such as liquid paraffin, isoparaffin, hexyl laurate, isopropyl myristate, myristyl myristate, cetyl myristate, 2-octyldodecyl myristate, isopropyl palmitate, 2-ethylhexyl palmitate; butyl stearate, decyl oleate, 2-octyldodecyl oleate, myristyl lactate, cetyl lactate, lanolin acetate, stearyl alcohol, cetostearyl alcohol, oleyl alcohol, avocado oil, almond oil, olive oil, cacao oil, jojoba oil, sesame oil, safflower oil, soy oil, camellia oil, squalane, persic oil, castor oil, mink oil, cotton seed oil, coconut oil, egg yolk oil, lard, and so forth; glycol ester oils such as polypropylene glycol monooleate, neopentyl glycol 2-ethylhexanoate, and so forth; polyhydric alcohol ester oils such as triisostearin, cocofatty acid triglycerides, and so forth; and polyoxyalkylene ether oils such as polyoxyethylene lauryl ether, polyoxypropylene cetyl ether, and so forth.

When the oil is preliminarily incorporated in the crosslinkable silicone composition, the quantity of oil incorporation must then be a quantity that exceeds the amount of oil that can be retained by the silicone powder provided by the crosslinking of the crosslinkable silicone composition, that is, the quantity of oil incorporation must be a quantity that exceeds the amount of oil that can be contained by this silicone powder. While the potential quantity of retention will vary with the particular oil/crosslinkable silicone composition combination, as a general matter the quantity of oil incorporation is preferably in the range from 200 to 5,000 weight parts per 100 weight parts of the crosslinkable silicone composition and particularly preferably is in the range from 250 to 2,000 weight parts per 100 weight parts of the crosslinkable silicone composition.

In a preferred method for producing the water-based emulsion of the silicone powder-containing oil composition, the crosslinkable silicone composition in which the oil has been preliminarily incorporated is emulsified in water followed by execution of the crosslinking reaction thereon. A stirring apparatus, e.g., a homomixer, paddle mixer, Henschel mixer, Homo Disper, colloid mill, propeller stirrer, homogenizer, inline continuous emulsifier, ultrasonic emulsifier, vacuum kneader/mixer, and so forth, can be used to emulsify the composition in water.

A surfactant, e.g., a nonionic surfactant, cationic surfactant, or anionic surfactant, is preferably used in order to effect a stable emulsification of the aforementioned composition in the water, and the use of a nonionic surfactant is preferred. The quantity of surfactant incorporation is preferably in the range from 0.1 to 20 weight parts per 100 weight parts of the oil-containing crosslinkable silicone composition and particularly preferably is in the range from 0.5 to 10 weight parts per 100 weight parts of the oil-containing crosslinkable silicone composition.

The average particle size of the oil droplets in the water-based emulsion of the silicone powder-containing oil composition prepared as described above is preferably in the range from 0.1 to 500 μm, more preferably in the range from 0.2 to 500 μm, even more preferably in the range from 0.5 to 500 and particularly preferably in the range from 0.5 to 200 μm. The reasons for this are as follows: it is quite difficult to prepare a water-based dispersion in which the average particle size of the oil droplets is below the lower limit for the above-cited range; a water-based dispersion in which the upper limit on the previously cited range is exceeded has a reduced stability.

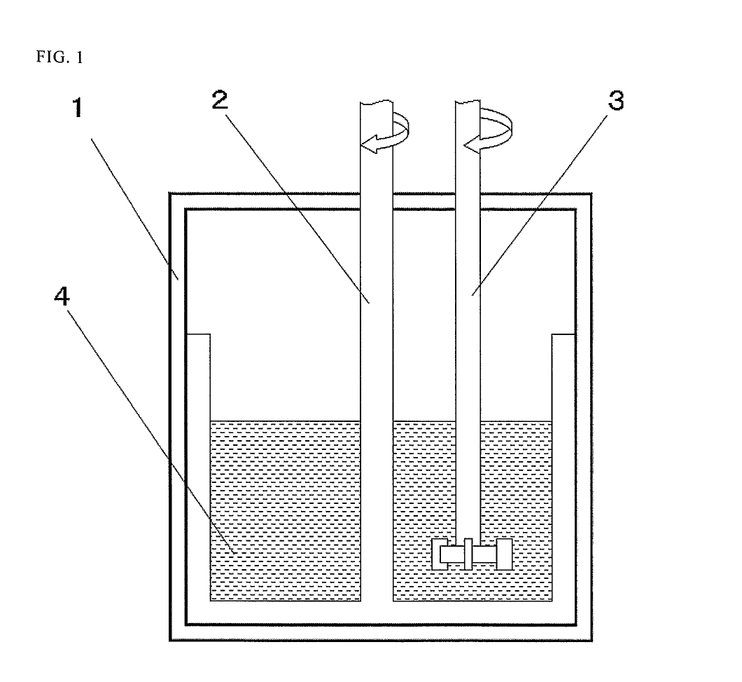

The production method of the present invention is characterized by the use—at the time of water removal while stirring the water-based emulsion of the silicone powder-containing oil composition under reduced pressure—of a stirring apparatus that has at least a stirring means that rotates at low speed along the inner wall of the container and a stirring means that rotates at high speed in the interior of the container. An example of a stirring apparatus that can be used by the present invention is shown in FIG. 1. The production method according to the present invention will be described in detail using FIG. 1.

A stirring means 2 stirs the water-based emulsion 4 as a whole within a container 1; this stirring is performed by low-speed rotation along the inner wall of the container. This stirring means 2 can be exemplified by an anchor mixer and by a scraper-equipped anchor mixer. This stirring means 2 inhibits local stagnation of the water-based emulsion within the container and thereby makes possible an even and uniform removal of the water from this emulsion. This stirring means 2 rotates at low speed, and its rotation rate is preferably in the range from 1 to 500 rpm.

The stirring means 3, through its high-speed rotation in the interior of the container 1, forcibly stirs the water-based emulsion therein or the oil composition therein provided by the removal of the water from the emulsion. The reasons for the preceding are as follows: the efficient removal of the water from the water-based emulsion is highly problematic when only the stirring means 2 is present; when only the stirring means 3 is present, the water-based emulsion 4 is stirred locally in the vicinity of the stirring means 3, and as a result the efficient removal of the water from the water-based emulsion 4 is highly problematic. This stirring means 3 can be exemplified by impeller-type stirring devices having a paddle or blade—e.g., a fan, propeller, soft cross, square cross, butterfly, turbine, disc turbine, curved disc turbine, blade turbine, tilted paddle, disperser, and so forth—mounted on a stirring shaft, and by homomixers comprising a high-speed rotating turbine blade and a stator. A disperser-mixer is particularly preferred. The stirring means 3 rotates at high speed, and its rotation rate is preferably in the range from 1,000 to 20,000 rpm.

Water is removed in the production method according to the present invention while stirring the water-based emulsion under reduced pressure; however, in order to accelerate water removal the emulsion is preferably heated to a temperature below 100° C. and particularly preferably is heated to a temperature in the range from 50 to 90° C. While the degree of pressure reduction is also not particularly limited, 100 mmHg and below is preferred and 50 mmHg and below is particularly preferred.

The oil composition obtained as described in the preceding contains a silicone powder uniformly dispersed in the oil, and its state can be exemplified by liquid, cream, paste, and grease.

### EXAMPLES

The method of the present invention for producing a silicone powder-containing oil composition will be described in detail using examples. The properties referenced in the examples are the values at 25° C. In addition, the average particle size of the emulsion, the average particle size of the silicone powder, and the properties of the oil composition were determined as follows.

### [The Average Particle Size of the Emulsion]

The emulsion was measured using an LA-750 laser diffraction particle size distribution analyzer from Horiba, Ltd. The median diameter provided by this measurement, that is, the particle diameter corresponding to 50% in the cumulative distribution, was taken to be the average particle size.

### [The Average Particle Size of the Silicone Powder]

The emulsion was air dried on a glass plate and the silicone powder was collected under a stereomicroscope to prepare the sample. This was observed under an electron microscope, and the average of 10 particle diameters was taken to be the average particle size.

### [Viscoelasticity of the Oil Composition]

The storage modulus G′ (Pa), the loss modulus G″ (Pa), and the loss tangent tan δ of the oil composition were measured using an ARES viscoelasticity analyzer from Rheometric Scientific, Inc. The measurement conditions were as follows: room temperature (25° C.), 25 mm parallel plates, gap: 0.5 to 0.6 mm, strain: 10%; oscillation rate: 0.01 to 50 Hz.

### [Viscosity of the Oil Composition]

The viscosity of the oil composition was measured using an EMD-type viscometer from Tokyo Keiki Inc. A 1.34°×R24 cone was used, and the value after 3 minutes at 50 rpm was recorded.

### Reference Example 1

A crosslinkable silicone composition was prepared by mixing the following: 9.13 weight parts of a dimethylpolysiloxane-methylvinylsiloxane copolymer endblocked by the dimethylvinylsiloxy group at both molecular chain terminals and having a viscosity of 400 mPa·s and a vinyl group content of 1.18 weight %, 0.87 weight part of a dimethylsiloxane-methylhydrogensiloxane copolymer endblocked by the trimethylsiloxy group at both molecular chain terminals and having a viscosity of 50 mPa·s and a silicon-bonded hydrogen content of 0.43 weight %, and 90 weight parts of a dimethylpolysiloxane endblocked by the trimethylsiloxy group at both molecular chain terminals and having a viscosity of 6 mPa·s.

To this composition, 29.5 weight parts of a previously prepared aqueous solution was added.(The aqueous solution was prepared by dissolving 1.6 weight parts of a polyoxyethylene alkyl ether with an HLB of 14.5 and 1.6 weight parts 2-phenoxyethanol into 96.8 weight parts pure water.) After emulsification with a colloid mill, an additional 27.6 weight parts of pure water was added to provide the water-based emulsion of a crosslinkable silicone composition.

To this emulsion was added, with mixing to uniformity, a water-based emulsion of a platinum-type catalyst having as its main component the 1,3-divinyl-1,1,3,3-tetramethyldisiloxane complex of platinum. The average particle size of the platinum-type catalyst in this water-based emulsion was 0.05 μm; the platinum metal concentration in this water-based emulsion was 0.05 weight %; and this water-based emulsion was added in a quantity that provided 10 weight-ppm platinum metal with reference to the crosslinkable silicone composition present in the emulsion.

The crosslinkable silicone composition was crosslinked by the hydrosilylation reaction, and the reaction was proceeded by holding the emulsion described above for 1 day at 50° C.Thereby the water-based emulsion of a silicone oil composition was obtained that contained a silicone rubber powder in silicone oil droplets that were themselves dispersed in the water.

### Reference Example 2

A crosslinkable silicone composition was prepared by mixing the following: 15.24 weight parts of a dimethylpolysiloxane endblocked by the dimethylvinylsiloxy group at both molecular chain terminals and having a viscosity of 400 mPa·s and a vinyl group content of 0.48 weight %, 4.76 weight parts of a dimethylsiloxane-methylhydrogensiloxane copolymer endblocked by the trimethylsiloxy group at both molecular chain terminals and having a viscosity of 75 mPa·s and a silicon-bonded hydrogen content of 0.05 weight %, and 80 weight parts of a dimethylpolysiloxane endblocked by the trimethylsiloxy group at both molecular chain terminals and having a viscosity of 6 mPa·s.

To this composition was added 29.5 weight parts of an aqueous solution prepared by dissolving 1.6 weight parts of a polyoxyethylene alkyl ether with an HLB of 14.5 and 1.6 weight parts of 2-phenoxyethanol in 96.8 weight parts of pure water. After emulsification with a colloid mill, an additional 27.6 weight parts of pure water was added to provide the water-based emulsion of a crosslinkable silicone composition.

To this emulsion was added the water-based emulsion of a platinum-type catalyst as used in Reference Example 1. Crosslinking of the crosslinkable silicone composition by the hydrosilylation reaction then produced the water-based emulsion of a silicone oil composition that contained a silicone rubber powder in silicone oil droplets that were themselves dispersed in the water.

### Reference Example 3

A crosslinkable silicone composition was obtained by mixing the following, cooled to 5° C., to uniformity: 9.05 weight parts of a dimethylpolysiloxane endblocked by the hydroxyl group at both molecular chain terminals and having a viscosity of 40 mPa·s and a hydroxyl group content of 3.8 weight %, 0.95 weight part of a methylhydrogenpolysiloxane endblocked by the trimethylsiloxy group at both molecular chain terminals and having a viscosity of 20 mPa·s and a silicon-bonded hydrogen content of 1.56 weight %, 90 weight parts of a dimethylpolysiloxane endblocked by the trimethylsiloxy group at both molecular chain terminals and having a viscosity of 6 mPa·s, and 0.10 weight part tin dioctylate.

To this composition was added 29.5 weight parts of an aqueous solution prepared by dissolving 1.6 weight parts of a polyoxyethylene alkyl ether with an HLB of 14.5 and 1.6 weight parts of 2-phenoxyethanol in 96.8 weight parts of pure water. After emulsification with a colloid mill, an additional 29.1 weight parts of pure water was added to provide the water-based emulsion of a crosslinkable silicone composition.

A condensation crosslinking reaction was carried out in the crosslinkable silicone composition by allowing the previously described emulsion to stand at quiescence for 1 week at room temperature, thereby producing the water-based emulsion of a silicone oil composition that contained a silicone rubber powder in silicone oil droplets that were themselves dispersed in the water.

### Reference Example 4

A crosslinkable silicone composition was prepared by mixing the following: 9.13 weight parts of a dimethylsiloxane-methylvinylsiloxane copolymer endblocked by the dimethylvinylsiloxy group at both molecular chain terminals and having a viscosity of 400 mPa·s and a vinyl group content of 1.18 weight %, 0.87 weight part of a dimethylsiloxane-methylhydrogensiloxane copolymer endblocked by the trimethylsiloxy group at both molecular chain terminals and having a viscosity of 50 mPa·s and a silicon-bonded hydrogen content of 0.43 weight %, and 90 weight parts Isozole 400 K(a isoparaffin represented by C₁₆H₃₄) from Nippon Petrochemicals Co., Ltd.

To this composition was added 29.5 weight parts of an aqueous solution in which 1.6 weight parts of a polyoxyethylene alkyl ether with an HLB of 14.5 and 1.6 weight parts of 2-phenoxyethanol were dissolved in 96.8 weight parts of pure water. After emulsification with a colloid mill, an additional 27.6 weight parts of pure water was added to provide the water-based emulsion of a crosslinkable silicone composition.

To this emulsion was added the water-based emulsion of a platinum-type catalyst as used in Reference Example 1. Crosslinking by the hydrosilylation reaction of the crosslinkable silicone composition then produced the water-based emulsion of an isoparaffin composition that contained a silicone rubber powder in isoparaffin droplets that were themselves dispersed in the water.

TABLE 1

| | Reference | Reference | Reference | Reference |
|---|---|---|---|---|
| | Example 1 | Example 2 | Example 3 | Example 4 |
| emulsion | | | | |
| average particle size | 2.5 | 2.4 | 1.9 | 2.3 |
| (μm) | | | | |
| stability | + | + | + | + |
| average particle size of | 2.4 | 2.3 | 1.5 | 1.9 |
| the silicone powder | | | | |
| (μm) | | | | |
| silicone powder/oil | 10/90 | 20/80 | 10/90 | 10/90 |
| weight ratio | | | | |

### Practical Example 1

1,700 g of the silicone oil composition water-based emulsion prepared in Reference Example 1 was introduced into a Model 3M-5 T.I. Combi Mix combination mixer from the PRIMIX Corporation. While stirring at an anchor mixer rotation rate of 80 rpm and a disperser mixer rotation rate of 1,000 rpm, the water was removed by reducing the pressure while raising the temperature to 75 to 85° C. over 2 to 3 hours and holding for 1 hour at 50 mmHg or below. This was followed by cooling to room temperature to obtain 1,070 g of a paste-form silicone oil composition having a water content of less than 0.1 weight %. When this silicone oil composition was observed with a stereomicroscope, the silicone rubber powder was found to be uniformly dispersed in the silicone oil and the shape of this silicone rubber powder was found to be spherical. The properties of this silicone oil composition are given in Table 2.

### Practical Example 2

100 g of the silicone oil composition water-based emulsion prepared in Reference Example 2 was introduced into a Model HV-030 Vacuum Mixer combination mixer from the SMT Co., Ltd. While stirring at an anchor mixer rotation rate of 90 rpm and a disperser mixer rotation rate of 1,000 rpm, the water was removed by reducing the pressure while raising the temperature to 75 to 85° C. over 1 to 2 hours and holding for 1 hour at 50 mmHg or below. This was followed by cooling to room temperature to obtain 60 g of a paste-form silicone oil composition having a water content of less than 0.1 weight %. When this silicone oil composition was observed with a stereomicroscope, the silicone rubber powder was found to be uniformly dispersed in the silicone oil and the shape of this silicone rubber powder was found to be spherical. The properties of this silicone oil composition are given in Table 2.

### Practical Example 3

55 g of a silicone oil composition that had a water content of less than 0.1 weight % was obtained by removing the water by the same procedure as in Example 2 from 100 g of the silicone oil composition water-based emulsion prepared in Reference Example 3. When this silicone oil composition was observed with a stereomicroscope, the silicone rubber powder was found to be uniformly dispersed in the silicone oil and the shape of this silicone rubber powder was found to be spherical. The properties of this silicone oil composition are given in Table 2.

### Practical Example 4

45 g of an isoparaffin composition that had a water content of less than 0.1 weight % was obtained by removing the water by the same procedure as in Example 2 from 100 g of the isoparaffin composition water-based emulsion prepared in Reference Example 4. This isoparaffin composition had a viscosity of 65 mPa·s and measurement of the viscoelasticity could thus not be performed under the conditions indicated above. When this isoparaffin composition was observed with a stereomicroscope, the silicone rubber powder was found to be uniformly dispersed in the isoparaffin and the shape of this silicone rubber powder was found to be spherical.

### Comparative Example 1

10 g of the silicone oil composition water-based emulsion prepared in Reference Example 1 was transferred to an aluminum dish with a diameter of 5 cm. Water removal was performed by air drying for 1 week in a draft to yield 6 g of a paste-form silicone oil composition that had a water content of less than 0.5 weight %. The properties of this silicone oil composition are given in Table 2. The silicone rubber powder in this silicone oil composition exhibited aggregation, and as a consequence this silicone oil composition exhibited a larger G′, a smaller G″, and a smaller tan 6 than did the silicone oil composition prepared in Example 1.

### Comparative Example 2>

100 g of the silicone oil composition water-based emulsion prepared in Reference Example 1 was introduced into a Model HV-030 Vacuum Mixer combination mixer from the SMT Co., Ltd. While stirring at an anchor mixer rotation rate of 90 rpm and a disperser mixer rotation rate of 1,000 rpm, the temperature was raised to 75 to 85° C. over 1 to 2 hours and this temperature was then held for 1 hour. This was followed by cooling to room temperature to obtain 96 g of a silicone oil composition that had a water content of greater than 30 weight %. The large water content in this silicone oil composition demonstrated that water removal had been inadequate.

TABLE 2

| viscoelasticity of the | | Practical | Practical | Practical | Comparative |
|---|---|---|---|---|---|
| oil composition | | Example 1 | Example 2 | Example 3 | Example 1 |
| G′ | 0.1 Hz | 731 | 603 | 87 | 1217 |
| | 1.0 Hz | 1035 | 732 | 99 | 1399 |
| G″ | 0.1 Hz | 435 | 86 | 12 | 213 |
| | 1.0 Hz | 495 | 176 | 17 | 232 |
| tan δ | 0.1 Hz | 0.59 | 0.14 | 0.14 | 0.17 |
| | 1.0 Hz | 0.48 | 0.24 | 0.17 | 0.17 |

### INDUSTRIAL APPLICABILITY

Because the silicone powder is uniformly dispersed in the oil in the oil composition provided by the production method of the present invention, this oil composition is well suited for use in lubricants, additives for resins and plastics, cosmetic materials, drugs and pharmaceuticals, and so forth.

### DESCRIPTION OF THE REFERENCE NUMBERS IN DRAWING

1 stirring apparatus container
2 stirring means that rotates at low speed
3 stirring means that rotates at high speed
4 water-based emulsion of a silicone powder-containing oil composition

1. A method of producing a silicone powder-containing oil composition, characterized by removing the water from a water-based emulsion of a silicone powder-containing oil composition while stirring the emulsion under reduced pressure with a stirring apparatus that has at least a stirring means that rotates at low speed along the inner wall of a container and a stirring means that rotates at high speed in the interior of the container.

2. The production method according to claim 1, wherein the silicone powder is a silicone rubber powder.

3. The production method according to claim 1, wherein the oil is a silicone oil or a silicon-free organic oil.

4. The production method according to claim 1, wherein the stirring means that rotates at low speed along the inner wall of the container is an anchor mixer.

5. The production method according to claim 1, wherein the stirring means that stirs at high speed in the interior of the container is a disperser mixer.

6. The production method according to claim 1, wherein the water is removed at a temperature below 100° C.

7. The production method according to claim 1, wherein the silicone powder is elastomeric and has an average particle size in the range from 0.05 to 100 μm.

8. The production method according to claim 1, wherein the low speed of the stirring means is in the range from 1 to 500 rpm.

9. The production method according to claim 1, wherein the high speed of the stirring means is in the range from 1,000 to 20,000 rpm.

10. The production method according to claim 8, wherein the high speed of the stirring means is in the range from 1,000 to 20,000 rpm.

11. The production method according to claim 1, wherein the reduced pressure is 100 mmHg and below.

12. The production method according to claim 10, wherein the reduced pressure is 100 mmHg and below.
